(19) United States
(12) Patent Application Publication
(10) Pub. No.: US 2021/0322627 A1
(43) Pub. Date: Oct. 21, 2021

(54) KITS, COMPOSITIONS, AND METHODS FOR VASCULAR EMBOLIZATION AND IMPLANTATION

(71) Applicant: Shienlin Saber, Laguna Beach, CA (US)

(72) Inventor: Shienlin Saber, Laguna Beach, CA (US)

(21) Appl. No.: 15/825,743

(22) Filed: Nov. 29, 2017

Related U.S. Application Data
(60) Provisional application No. 62/413,393, filed on Oct. 26, 2016.

(51) Int. Cl. A61L 24/04; A61L 24/02; A61L 24/00

(52) U.S. Cl. CPC A61L 24/046; A61L 24/02; A61L 24/0015; C08L 83/04; A61L 2400/06; A61L 2430/36; A61L 2300/418

(57) ABSTRACT

The inventive subject matter provides compositions and methods for vascular embolization and implantation. Contemplated compositions are polymerizable in situ in the presence of blood, and can optionally be delivered to form a cured stent or embolus.

This application claims priority to US provisional application with the Ser. No. 62/413,393 which was filed Oct. 26, 2016

### FIELD OF THE INVENTION

The field of the invention is vascular embolization and implantation.

### BACKGROUND

The background description includes information that may be useful in understanding the present invention. It is not an admission that any of the information provided herein is prior art or relevant to the presently claimed invention, or that any publication specifically or implicitly referenced is prior art.

Benign and malignant tumors rely on blood supply to grow and metastasize. Embolization is a minimally invasive procedure that could occlude or close off specific vessels that are supplying blood to a tumor, especially when the tumor is difficult or impossible to remove, to shrink or reduce the growth rate of the tumor.

When surgical removal of the tumor is possible, difficulties can occur relating to excessive bleeding and vascular episodes. These difficulties are sometimes exacerbated due to the inability to visualize the tumor or blood vessels, which can lead to increased surgical times.

Some known efforts have been placed towards reducing some of these risks during surgical procedures. For example, Gelfoam® is a sterile compressed sponge that can be used as a hemostatic device capable of absorbing up to 45 times its weight in blood. Unfortunately, vessels embolized with Gelfoam have been said to recanalize within a short amount of time, with the recanalization being notoriously unpredictable with respect to degree and timing.

Onyx® is a non-adhesive liquid embolic agent comprised of ethylene vinyl alcohol (EVOH) dissolved in dimethyl sulfoxide (DMSO), which is often used for pre-surgical embolization. Upon contact with blood, the DMSO diffuses out and the EVOH polymerizes. Unfortunately, systemic toxicity has been noted as a major concern with the use of Onyx because of the action potential reducing effects of DMSO.

As another example, LeGoo™ is a biopolymer gel that allows surgeons to temporarily stop blood flow in a vessel during surgery without the use of a clamp or other conventional occlusion device. LeGoo is a liquid gel at colder temperature, and forms a plug when injected into a blood vessel. The plug dissolves via cooling (or spontaneously after several minutes), and cannot subsequently reform. Unfortunately, LeGoo is only marketed for temporary endovascular occlusion of blood vessels up to 4 mm in diameter, and is apparently unsuitable for long term embolization.

Thus, there is still a need for improved kits, compositions and methods for vascular embolization and implantation.

### SUMMARY OF THE INVENTION

The inventive subject matter comprises kits, compositions and methods for placement of a polymerizable composition within a target site of a vessel (e.g., arteries, capillaries and vein), for example as an embolic agent or a stent.

Some preferred polymerizable compositions are silicone elastomer compositions comprising two or more separate components. A first component can include a catalyst and a second component can include a cross-linker such that when the two components are combined or mixed, a temporary or permanent seal quickly forms and adheres to the inner wall of the vessel. In some contemplated aspects, a therapeutically effective amount of a drug or treatment composition can be suspended or otherwise incorporated into one or more components of the polymerizable formulation. For example, a drug having a sustained release property can be incorporated into the polymerizable composition, and can be effective to deliver the drug to an area near the attachment site over a prolonged period of time.

The polymerizable composition can have a work time sufficient such that the two or more components can be delivered simultaneously to the target site via a catheter without clogging. Where desired or necessary, the two or more components of the polymerizable compositions can be delivered to the target site sequentially to further prevent unwanted and premature curing within the catheter.

In some aspects, a base composition could be delivered to the target site prior to the polymerizable composition. The base component could prevent or block a flow of the polymerizable composition, enhance an adhesion of the polymerizable composition to an inner wall of the vessel, or minimize an adhesion of the polymerizable composition to a catheter delivering the polymerizable composition. For example, the base composition could comprise a composition that polymerizes upon exposure to blood or the vessel outside of the catheter to form a temporary seal that spontaneously or otherwise dissolves. The temporary seal could block a flow of the polymerizable composition until the more permanent seal is formed, and subsequently be dissolved.

Contemplated polymerizable compositions, upon forming a seal (e.g., stent, embolus) in situ, may be inelastic, elastic or semi-elastic. For example, some contemplated compositions for forming an embolus can have an elasticity at break of between 120-1000% (e.g., between 400 and 800% or between 450-600%) and mimic the elasticity of the target site to reduce the likelihood of dislodgment or tear. Some contemplated compositions for forming a stent can have the same, a greater inelasticity, or a reduced inelasticity.

Applicant has surprisingly discovered that the presence of blood could in fact improve (e.g., accelerate) a cure time of the polymerizable composition. When some contemplated formulations were placed on an injury where blood is exposed, the cure time was found to decrease by about 15 seconds (compared to cure time on skin without the presence of blood). Without wishing to be bound by any particular theory, the Applicant contemplates that the temperature of the blood and its iron content may speedup the cure time of the polymerizable formulation. Additionally, the polymerizable composition has been found to adhere to the tissue even in the presence of blood, and infuse approximately 0.25 mm-5 mm into surrounding tissue and vessels. It is contemplated that the polymerizable composition could infuse further into the tissue and vessels, for example up to 1 foot or even further. Therefore, it is contemplated that a secure seal could be provided at a target site of a vessel, optionally without causing an embolus where desired (e.g., when implanting a stent). The polymerizable composition can be removed with the removal of the tumor or arteriovenous malformations where desired.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates a dual syringe used to store and dispense the polymerizable formulation.

FIGS. 2A-2C illustrate injection of polymerizable formulations into tubing and removal from the tubing.

FIGS. 3A-3E illustrate a formulation similar to that of FIG. 2A injected into and removed from a cardiac vessel.

### DETAILED DESCRIPTION

The inventive subject matter provides compositions and methods for placement of a polymerizable composition within a vessel to form a cured seal in situ, for example an embolus or a stent. As used herein, the term “cured seal” should be interpreted broadly to include any material formed by a polymerizable composition that can searingly contact or adhere to an inner wall of a vessel.

Contemplated seals can be partial or complete, and can increase or decrease blood flowing through the target site of the vessel. For example, a complete seal can decrease or even block a blood flow through a target site (e.g., forming an embolus), and a partial seal could expand a diameter of a blood vessel to increase blood flow through a target site (e.g., forming a stent). It is further noted that the terms ‘vessel’ and ‘blood vessel’ are used interchangeably herein, and that contemplated vessels include arterial and venous vessels as well as capillary vasculature. Thus, a vessel will typically have a diameter of at least 0.5 mm, and more typically at least 1 mm.

Additionally or alternatively, contemplated seal scan comprise a short term temporary seal, a long term temporary seal, or a permanent seal. For example, it is contemplated that a seal can remain adhered to an inner or outer surface of a vessel (or a stent placed in the vessel) for a period of at least 1 hour (e.g., at least 3 hours, at least 5 hours, at least 8 hours, at least 10 hours, at least 12 hours, at least 15 hours, at least 20 hours, at least 1 day, at least 2 days, at least 3 days, at least 4 days, at least 5 days, at least 1 month, at least 1 year, between 1-10 days, between 1-10 weeks, between 1-10 years, or even longer). Viewed from a different perspective, it is contemplated that a seal formed on the blood vessel can be configured to remain substantially adhered to the substrate (e.g., at least 80% of the seal remains adhered to the vessel or stent, at least 90% of the seal remains adhered to the vessel or stent, at least 95% of the seal remains adhered to the vessel or stent, 100% of the seal remains adhered to the vessel or stent) for a period of at least 1 hour (e.g., at least 3 hours, at least 5 hours, at least 8 hours, at least 10 hours, at least 12 hours, at least 15 hours, at least 20 hours, at least 1 day, at least 2 days, at least 3 days, at least 4 days, at least 5 days, at least 1 month, at least 1 year, between 1-10 days, between 1-10 weeks, between 1-10 years, or even longer).

In some preferred aspects, the polymerizable composition will result in a cured seal having sufficient elasticity to allow the user to move around comfortably without dislodging the seal or damaging the blood vessel. In other preferred aspects, the polymerizable composition can be introduced into a vessel and removed without causing damage to the vessel. If desired, it is also contemplated that the cured seal can be left in the vessel without causing damage to the patient. The polymerizable composition is typically odorless, non-toxic, hypoallergenic, compatible with other treatments, bacteriostatic, non-temperature sensitive, and removable as a single piece, or in sections, after curing.

While polymerizable composition can comprise a one part system, for example, where the polymerizable composition is temperature or light activated, preferred polymerizable composition can comprise a multi-part system, for example, where a catalyst and a cross-linking component must be separated to prevent premature and undesired curing.

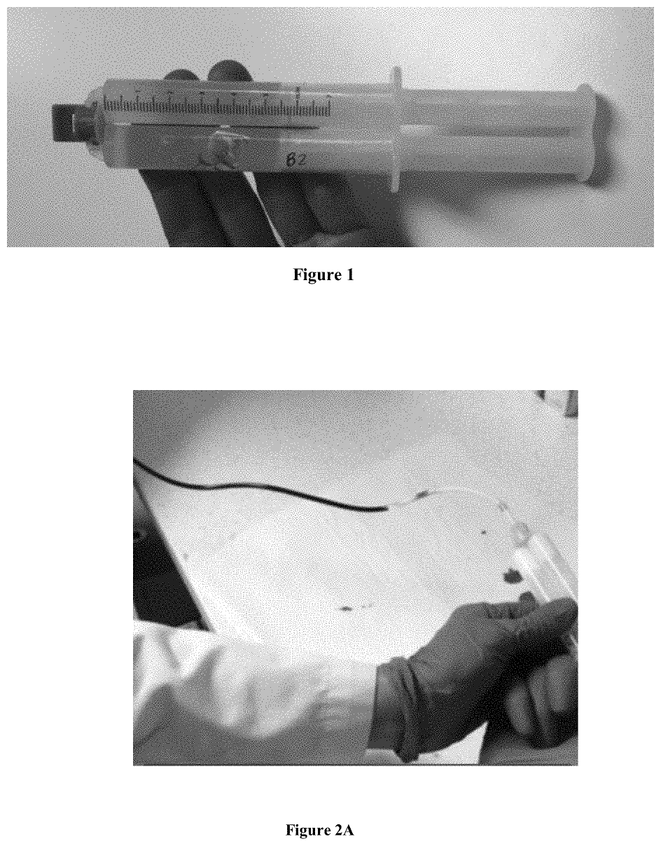

One type of a preferred polymerizable composition comprises a two-part elastomer system that cures at room or body temperature and includes (a) a first formulation including a polymer and catalyst (e.g., siloxane polymer and platinum catalyst), and (b) a second formulation comprising a polymer and a crosslinker. One or both of the formulations could include one or more of a filler, a thixotropic agent, an adhesion promoter, and a cure inhibitor to control the cure kinetics.

Where the first and second formulations are separately packaged or contained in a dual chambered syringe (see FIG. 1), crosslinking cannot occur until the two components are mixed together (e.g., delivered separately via a catheter to the target site, or delivered as a mixture).

In some contemplated embodiments, the polymer is a silicone polymer (siloxane polymer) with a polymer backbone of alternating silicone and oxygen atoms (i.e., siloxane bonds), and hydrocarbon (saturated, unsaturated, aromatic) organic side groups such as methyl, phenyl or vinyl, or a hydrogen attached to the silicon atoms. The siloxane polymer can comprise between 20-100 wt %, more preferably at least 50 wt %, and even more preferably at least 70 wt % (e.g., between 75-85 wt %, between 78-82 wt %) of the polymerizable composition (i.e., of the combined two part formulation where the catalyst and crosslinker are combined).

Where PDMS is used, it can be a linear polymer made up of repeating Si—O—Si linkages and a reactive vinyl group on both ends of the polymer chain. There may be organic side groups such as dimethyl bonded to every silicone molecule the backbone of the polymer. Siloxane polymers can also be substituted with diphenyl, methylphenyl, trifluoropropyl, or any combination thereof. Some exemplary siloxanes include oligosiloxanes, polydimethylsiloxane (PDMS), vinyl-endblocked polydiphenyl siloxane, vinyl-endblocked polymethylphenylsiloxane, vinyl-endblocked trifluoropropyl siloxane, vinyl-endblocked polydiethyl siloxane, trimethyl-endblocked methylvinyl polydimethylsiloxane, trimethyl-endblocked methylvinyl polydiphenylsiloxane, trimethyl-endblocked methylvinyl polymethylphenylsiloxane, trimethyl-endblocked methylvinyl polytrifluoropropylsiloxane, and trimethyl-endblocked ethylvinyl polydimethylsiloxane. Contemplated siloxanes can be optically clear, non-toxic and non-flammable.

All suitable chain lengths of the siloxane polymer are contemplated, including between 10-2,500 repeating units long, between 200-1,000 repeating units long, or between 300-400 repeating units long (e.g., 340-360), which equates to a molecular weight of ˜26,000 Daltons.

According to another embodiment, a polymer can include a main chain formed primarily of organosiloxane units. Among the silicone compounds contemplated, some may display both curing and adhesive properties, for example depending on the proportion of silicone or whether they are used with a particular additive. It may therefore be possible to adjust the properties of said compositions according to the proposed use.

In some contemplated embodiments where the polymer is a siloxane, the crosslinker is a siloxane crosslinker such as a methyl-hydrogen crosslinker. The crosslinker can comprise between 0.1-50 wt %, between 0.1-10 wt %, and more preferably between 1-5 wt % (e.g., 2 wt %) of the polymerizable composition. An exemplary siloxane crosslinker used in some contemplated compositions is a small chain polymer that is trimethyl endblocked, making the ends of the chain non-functional. All suitable chain lengths of the crosslinker are contemplated, including for example, between 1-100 repeating units, more preferably between 1-50 units, and more preferably between 5-15 units (e.g., 10 units wherein the molecular weight is 800 Daltons). Along the backbone of the crosslinker can be reactive methyl-hydrogen side groups which can comprise between 1-99 mole %, more preferably between 20-80 mole %, and more preferably between 40-60 mole % (e.g., 50 mole %) of the crosslinker. The remaining mole % can comprise dimethyl side groups. Where each of the methyl-hydrogen side groups and the dimethyl side groups make up approximately 50 mole %, approximately half of the repeating units of the crosslinker will be dimethyl, and approximately half will be methyl hydrogen.

Other contemplated crosslinkers include hydride-endblocked polydimethylsiloxane, hydride-endblocked methylhydrogen polysiloxane, trimethyl-endblocked methylhydrogen methylvinyl polysiloxane, trimethyl-endblocked 100 mole % methylhydrogen polysiloxane, hydride-endblocked polydiphenylsiloxane, and hydride-endblocked phenylhydrogen polysiloxane.

Although the exemplary crosslinkers described above are siloxane crosslinkers, it should be appreciated that a person skilled in the art would be able to select a suitable crosslinker based on the polymer included in the polymerizable compositions.

The catalysts of contemplated polymerizable formulations can comprise a peroxide, platinum, tin, a combination thereof, or other suitable catalyst. An exemplary platinum catalyst for hydrosilylation reactions can comprise a complex of platinum with a vinyl siloxane acting as a ligand. An example of this is the Karstedt's catalyst. Other contemplated catalysts include, rhodium complex in vinyl silicone fluid, organotin catalyst such as dibutyltin dilaurate, stannous octoate, dibutlytin diacetate, peroxide catalysts such as benzoyl peroxide, 2,4 dichlorobenzoyl peroxide, dicumyl peroxide, 2,5-Dimethyl-2,5-di(tert-butylperoxy)hexane.

The catalyst can be present in the formulation in any suitable amount, for example, between 0.001-10 wt % (of the combined two part formulation where the catalyst and crosslinker are combined), more preferably between 0.01 and 1 wt %, and more preferably between 0.07 and 0.13 wt % (e.g., 0.1 wt %) of the polymerizable composition, and can include between 1-250 ppm, between 5-70 ppm, more preferably between 15-60 ppm (e.g., 30 ppm) of pure platinum.

The platinum catalyst will preferably be separated from the crosslinker until placed within, or on, the injury. Alternatively or additionally, the platinum catalyst can be combined with the crosslinker no more than 10 minutes, no more than 5 minutes, no more than 3 minutes, and preferably no more than 1 minute or 0.5 minute prior to being placed within, or on, the injury. Alternatively or additionally, the component or formulation comprising the platinum catalyst can be placed within the injury before or after the formulation or component comprising the crosslinker is placed within, or on, the injury. As discussed in more detail below, a base component (e.g., spray) comprising the same or different catalyst could be applied prior to any of the first formulation (including the platinum catalyst) and the second formulation (including the crosslinker).

Where a filler is included in the polymerizable formulation, an exemplary filler includes amorphous fumed silica having a surface area of between 100-300 m²/gram (e.g., approximately 200 m²/gram). Other contemplated fillers include fumed silica with low surface area (e.g., 100 m²/gram), fumed silica with high surface area (e.g., 400 m²/gram), precipitated silica, diatomaceous earth, titanium dioxide, zinc oxide, barium sulfate, colloidal silica, and boron nitride.

The filler can comprise between 0-80 wt %, more preferably between 5-35 wt % and even more preferably between 10-23 wt % (e.g., 16 wt %) of the combined two part formulation where the catalyst and crosslinker are combined. The surface of the silica can be treated with trimethyl silyl groups so that it is more soluble with the polymer.

A suitable thixotropic additive (e.g., a compound that reduces the flowability of a material rendering it non-slump) can also be included in some contemplated polymerizable compositions in any suitable amount. For example, the thixotrope can comprise between 0.1-5 wt %, between 0.5-2.5 wt %, and more preferably between 1-2 wt % (e.g., 1.5 wt %) of the combined two part formulation where the catalyst and crosslinker are combined. An exemplary thixotrope included in some contemplated formulations is a hydroxyl endblocked polydimethyl siloxane with a chain length of between 10-20 repeating units (e.g., 15 repeating units with a molecular weight of 1100 Daltons). The hydroxyl groups on the polymer ends can react with the surface hydroxyl groups of the fumed silica causing the silica to become less flowable.

Suitable adhesion promoters can also be included in the polymerizable composition to increase the bond strength of the adhesive (polymerizable composition or seal) to the substrate (inner wall of the vessel) as curing occurs. Tetrapropoxysilane is an exemplary adhesion promoter commonly used in silicone primers. Without wishing to be bound by any particular theory, the applicant contemplates that the reactive silane may form hydrogen or even covalent bonds with the vessel. The adhesion promoter, when included in the polymerizable composition, can comprise between 0.01-10 wt %, between 0.1 and 5 wt %, and more preferably between 0.4 and 1.2 wt % (e.g., 0.8 wt %) of the polymerizable composition.

Additional adhesion promoters suitable for contemplated polymerizable formulations include those shown in Table 1. Equal parts of the first and second components including the different adhesion promoters were mixed and a thin layer was applied to a forearm and allowed to vulcanize at room temperature. The samples were evaluated by recording the time that the edges began to lift from the skin. Once the edges lifted, the samples were peeled off and evaluated qualitatively for how difficult it was to peel complete off the skin. Each adhesion promoter was evaluated, and the results are described in Table 1 below. All percentages used herein are weight percentages (wt %) unless otherwise indicated. Although the adhesions promoters described below were tested on skin, it is contemplated that the formulations would have even stronger adhesions to the blood vessel based on tests performed using animal tissue and vessels in the presence of blood.

TABLE 1

| | Edge Lifting | | Adhesion |
|---|---|---|---|
| Formulation | Began | | to skin |
| Formulation of table 2 with no | 1 | hour | poor |
| adhesion promoter | | | |
| Formulation of table 2 with 1% | 5 | hours | good |
| Tetrapropoxysilane | | | |
| Formulation of table 2 with 1.5% | 5 | hours | good |
| Tetrapropoxysilane | | | |
| Formulation of table 2 with 1% | 3.25 | hours | good |
| 3-aminopropropyltrimethoxysilane | | | |
| Formulation of table 2 with 1.5% | 3.25 | hours | good |
| 3-aminopropropyltrimethoxysilane | | | |
| Formulation of table 2 with 1% | 2 | hours | poor |
| Tris(2-methoxyethoxy)(vinyl)silane | | | |
| Formulation of table 2 with 1.5% | 2.5 | hours | poor |
| Tris(2-methoxyethoxy)(vinyl)silane | | | |
| Formulation of table 2 with 1% | 2 | hours | poor |
| Vinyltriethoxysilane | | | |
| Formulation of table 2 with 1.5% | 2.5 | hours | poor |
| Vinyltriethoxysilane | | | |
| Formulation of table 2 with 1% | 4 | hours | good |
| Tetrakis(2-methoxyethyl)ester | | | |
| Formulation of table 2 with 1% | 4 | hours | good |
| Tetrakis(2-methoxyethyl)ester | | | |
| Formulation of table 2 with 1% | 5 | hours | good |
| Thmethoxy-7-octenylsilane | | | |
| Formulation of table 2 with 1% | 5 | hours | good |
| Thmethoxy-7-octenylsilane | | | |
| Formulation of table 2 with 0.25% | 6.5 | hours | good |
| N-(triethoxysilylpropyl)-O- | | | |
| polyethylene oxide urethane | | | |
| Formulation of table 2 with 0.5% | 6.5 | hours | good |
| N-(triethoxysilylpropyl)-O- | | | |
| polyethylene oxide urethane | | | |
| Formulation of table 2 with 0.25% | 18 | hours | Excellent |
| N-(triethoxysilylpropyl)-O- | | | (synergistic effect |
| polyethylene oxide urethane | | | with respectto |
| and 1.5% Tetrapropoxysilane | | | adhesion where |
| | | | two adhesion |
| | | | promoters |
| | | | were used) |

The formulation used in each of the formulations of Table 1 are shown in Table 2. The adhesion promoter(s) of Table 1 were added to Part 2 of the formulation. However, it should be appreciated that the adhesion promoter could alternatively or additionally be added to Part 1 of the formulation. It should also be appreciated that the percentages shown in Part 2 below are modified once the adhesion promoter(s) are added.

TABLE 2

| Part 1 | | Part 2 | |
|---|---|---|---|
| Component | Wt % | Component | Wt % |
| Vinyl endblockedpolydimethyl | 79.95 ± 5 | Vinyl endblockedpolydimethyl | 76.985 ± 5 |
| siloxane polymer | | siloxane polymer | |
| Fumedsilica with surface | 19.99 ± 2 | Fumedsilica with surface | 19.2 ± 2 |
| area of 200 m²/gram | | area of 200 m²/gram | |
| Platinum catalyst complex | 0.06 ± .3 | Trimethyl endblockedmethyl- | 3.8 ± 1 |
| | | hydrogen siloxane polymer | |
| | | crosslinker (containing 50% | |
| | | methyl hydrogen and 50% dimethyl) | |
| | | 1,3,5,7-tetramethyl- | 0.015 ± .005 |
| | | 1,3,5,7-tetravinyl- | |
| | | cyclotetrasiloxane | |

It is also contemplated that inert pigments can be suspended in the polymerizable formulations without leaching. The inert pigments could advantageously allow a surgeon or physician to visualize the tumor while surgically removing it. The pigments used could contrast with blood and surrounding tissue to allow for faster and more effective removal of the tumor and improved outcome for the patient. Some contemplated powdered pigments can advantageously be broken down to a size of less than 20 microns, more preferably less than 15 microns to allow for even distribution or dispersion throughout the polymerizable formulation. Additionally or alternatively, concentrated liquid or gum color pigments can be added to one or more components of the polymerizable formulation.

Radio opaque or other particles (e.g., barium sulfate, zirconium dioxide) could be suspended or otherwise incorporated into the polymerizable formulations such that the cured seal can be detected by X-ray, computed tomography scans, ultrasound imaging or MRI scans during the embolization or implantation procedure. In some preferred embodiment, at least 8 wt %, more preferably at least 10 wt % (e.g., at least 11 wt %, between 8-50 wt %, between 10-20 wt %) is included in the combined two part polymerizable formulation for detection by X-ray. The radio opaque particles could be added to the polymerizable formulation in any commercially suitable matter, and could even be pre-mixed with one or more of its components. For example, the radio opaque particles could be mixed in with the first formulation component, second formulation component, a silicone polymer, a platinum catalyst, a crosslinker, an adhesion promoter, a cure inhibitor, a filler, a thixotropic agent, or any combination thereof.

It should be appreciated that the polymerizable formulations presented herein provide several advantageous effects including the potential to reduce the time required for tumor removal, enhance visualization of the tumor and surrounding vital structures prior to, during and after surgery, and provide a non-toxic seal whose mechanical and chemical properties can be modified pre-cure to provide a temporary or permanent polymerizable implant within selected blood vessels.

It is contemplated that the first and second formulations can react with each other at various temperatures, including for example at temperatures between −20 and 80 degrees Celsius, more typically between 0 and 60 degrees Celsius, and even more typically between 10 and 50 degrees Celsius, or around 35-40 degrees Celsius (body temperature). For example, it is contemplated that the formulations will be capable of reacting together to form a seal within a blood vessel and in the presence of blood by a hydrosilylation reaction or a condensation reaction, or a crosslinking reaction in the presence of a peroxide.

A complete seal can be formed within 20 minutes, within 10 minutes, more preferably within five minutes, within three minutes, within two minutes, or even within one minute. The seal can be any suitable size and shape, depending on the type of vascular disease.

Upon full curing, the seal can have a hardness sufficient to prevent unwanted flowing of the seal. For example, the seal can have a hardness of at least 10 on the Shore 00 durometer scale, at least 10 on the Shore A scale, a hardness of between 0 on the Shore 00 durometer scale and 40 on the Shore A durometer scale, a hardness of between 10 on the Shore 00 durometer scale and 30 on the Shore A durometer scale, a hardness of between 15-25 on the Shore A durometer scale, or a hardness of between 18-22 on the Shore A durometer scale. The work time of the polymerizable composition can be approximately half of the cure time (e.g., about sixty seconds where the cure time is about two minutes).

Additionally or alternatively, the seal can have an elasticity that allows for movement and stretching of the blood vessel without compromising the seal or causing damage to the vessel. For example, some contemplated compositions will have an elasticity at break of at least 120%, more preferably at least 200%, and more preferably at least 250% (e.g., between 120-1000%, between 400 and 800%, between 450-600%). As used herein, the term “% elasticity at break” refers to the extension of a length of a cured seal from an unstretched and normal configuration before tearing, at room temperature, wherein the cured seal has a thickness of between 3-5 mm in the unstretched, normal configuration. For example, where a cured seal has an at least 180% elasticity at break, the cured seal, when normally having a thickness of between 1-25 mm, can be stretched to at least 180% of its length before tearing (e.g., from 10 mm to at least 18 mm before tearing). Viewed from a different perspective, the seal can have a tensile strength that allows significant force to be applied while maintaining its integrity (e.g., between 100-2000 psi, between 200-800 psi, between 400-650 psi).

### EXAMPLES

Table 3 shows an exemplary two part polymerizable formulation having a dual adhesion promoter system. The two adhesion promoters work synergistically to increase adhesion to skin or tissue when compared to formulations having only one of the adhesion promoters. Without wishing to be bound by any particular theory, Applicant contemplates that one adhesion promoter makes the second more available at the surface of the formulation. Although the two adhesion promoters in this example are provided in Part 2 of the formulation, it should be appreciated that one adhesion promoter could be provided in each of Parts 1 and 2, that both adhesion promoters could be provided in Part 1, or that one adhesion promoter could be provided in the polymerizable formulations while a second adhesion promoter is provided in a base component.

The formulation of Table 1 has a working time of between 20-40 seconds (typically about 30 seconds), and a setting time of between 4-6 minutes (typically about 5 minutes) when Part 1 and Part 2 are mixed together and placed on skin. Based on experiments wherein the formulation was tested on animal tissue in the presence of blood, Applicant expects similar or even better results in blood vessels.

TABLE 3

| Part 1 | | Part 2 | |
|---|---|---|---|
| Component | Wt % | Component | Wt % |
| Vinyl endblockedpolydimethyl | 79.947 | Vinyl endblockedpolydimethyl | 74-75 |
| siloxane polymer (chain length | | siloxane polymer (chain length | |
| 350 repeating units) | | 350 repeating units) | |
| Fumedsilica with surface | 19.91 | Fumedsilica with surface | 18.5-19 |
| area of 200 m²/gram | | area of 200 m²/gram | |
| Platinum catalyst complex | 0.143 | Trimethyl endblockedmethyl- | 3.5-4.0 |
| | | hydrogen siloxane polymer | |
| | | crosslinker (containing 50 wt % | |
| | | methyl hydrogen and 50 wt % dimethyl) | |
| | | 1,3,5,7-tetramethyl- | 0.013-0.017 |
| | | 1,3,5,7-tetravinyl- | |
| | | cyclotetrasiloxane | |
| | | Tetrapropoxysilane | 2.2-2.8 |
| | | adhesion promoter | |
| | | N-(triethoxysilylpropyl)-O- | 0.37-0.45 |
| | | polyethylene oxide urethane | |
| | | adhesion promoter | |

It is also contemplated that the components shown in Table 1 could be included in Part 1 and Part 2 of the formulation in different concentration ranges as set forth below in Table 4 with comparable work times (e.g., between 10-120 seconds), setting times (e.g., between 1-10 minutes), adhesion properties (as described in Table 1), hardness of between 5-80 on the Shore A hardness scale, tensile strength between 200-1500 psi, and elasticities at break (of between 200-1000%).

TABLE 4

| Part 1 | | Part 2 | |
|---|---|---|---|
| Component | Wt % | Component | Wt % |
| Vinyl endblockedpolydimethyl | 60-90 | Vinyl endblockedpolydimethyl | 60-90 |
| siloxane polymer (100-1000 DP) | | siloxane polymer (100-1000 | |
| | | repeating siloxy units) | |
| Fumedsilica with surface | 10-30 | Fumedsilica with surface | 10-30 |
| area of 200 m²/gram | | area of 200 m²/gram | |
| (100-400 m²/gram) | | | |
| Platinum catalyst complex | 0.06-0.2 | Trimethyl endblockedmethyl- | 2-10 |
| | | hydrogen siloxane polymer | |
| | | crosslinker (containing 50% | |
| | | methyl hydrogen and 50% dimethyl) | |
| | | 1,3,5,7-tetramethyl- | .001-.05 |
| | | 1,3,5,7-tetravinyl- | |
| | | cyclotetrasiloxane | |
| | | Tetrapropoxysilane | 1-5 |
| | | adhesion promoter | |
| | | N-(triethoxysilylpropyl)-O- | 0.1-2 |
| | | polyethylene oxide urethane | |
| | | adhesion promoter | |

Table 5 shows an exemplary formulation including barium sulfate, which is contemplated to have comparable work times (e.g., between 10-120 seconds), setting times (e.g., between 1-10 minutes), adhesion properties, hardness, tensile strength, and elasticities at break as the formulation of Table 1.

TABLE 5

| Part 1 | | Part 2 | |
|---|---|---|---|
| Component | Wt % | Component | Wt % |
| Vinyl endblockedpolydimethyl | 66.6 ± 20 | Vinyl endblockedpolydimethyl | 74.9 ± 20 |
| siloxane polymer | | siloxane polymer | |
| Fumedsilica with surface | 16.7 ± 10 | Fumedsilica with surface | 18.7 ± 10 |
| area of 200 m²/gram | | area of 200 m²/gram | |
| Platinum catalyst complex | 0.05 | Trimethyl endblockedmethyl- | 3.7 ± 3 |
| | (0.001-0.2) | hydrogen siloxane polymer | |
| | | crosslinker (containing 50% | |
| | | methyl hydrogen and 50% dimethyl) | |
| Barium Sulfate (to make | 16.7 ± 15 | 1,3,5,7-tetramethyl- | 0.015 |
| the formulation radio- | | 1,3,5,7-tetravinyl- | (between |
| opaque) | | cyclotetrasiloxane | 0.001-0.05) |
| | | Tetrapropoxysilane | 2.2 ± 2 |
| | | adhesion promoter | |
| | | N-(triethoxysilylpropyl)-O- | 0.37 |
| | | polyethylene oxide | (0.1-2) |
| | | urethane adhesion | |
| | | promoter | |

Table 6 shows an exemplary formulation including one or more pigments, which is contemplated to have comparable work times (e.g., between 10-90 seconds), setting times (e.g., between 1-10 minutes), adhesion properties, and elasticities at break as the formulation of Table 1.

TABLE 6

| Part 1 | | Part 2 | |
|---|---|---|---|
| Component | Wt % | Component | Wt % |
| Vinyl endblockedpolydimethyl | 78.4 ± 20 | Vinyl endblockedpolydimethyl | 74.9 ± 20 |
| siloxane polymer | | siloxane polymer | |
| Fumed silica with surface | 19.6 ± 10 | Fumedsilica with surface | 18.7 ± 10 |
| area of 200 m²/gram | | area of 200 m²/gram | |
| Platinum catalyst complex | 0.06 | Trimethyl endblockedmethyl- | 3.7 ± 3 |
| | (0.001-0.2) | hydrogen siloxane polymer | |
| | | crosslinker (containing 50% | |
| | | methyl hydrogen and 50% dimethyl) | |
| Pigment (e.g., yellow, | 1.96 ± 1.5 | 1,3,5,7-tetramethyl- | 0.015 |
| orange, green, blue, brown) | | 1,3,5,7-tetravinyl- | (between |
| | | cyclotetrasiloxane | 0.001-0.05) |
| | | Tetrapropoxysilane | 2.2 ± 2 |
| | | adhesion promoter | |
| | | N-(triethoxysilylpropyl)-O- | 0.37 |
| | | polyethylene oxide | (0.1-2) |
| | | urethane adhesion | |
| | | promoter | |

Tables 7-10 show exemplary formulations only including one adhesion promoter, which is contemplated to have comparable work times (e.g., between 10-90 seconds), setting times (e.g., between 1-10 minutes), hardness, tensile strength, and elasticities at break as the formulation of Table 1, but a lower adhesion strength to skin (and tissue/vessels) likely due to a lack of synergistic effect with a second adhesion promoter.

TABLE 7

| Part 1 | | Part 2 | |
|---|---|---|---|
| Component | Wt % | Component | Wt % |
| Vinyl endblockedpolydimethyl | 79.95 ± 20 | Vinyl endblockedpolydimethyl | 75.29 ± 20 |
| siloxane polymer | | siloxane polymer | |
| Fumed silica with surface | 19.99 ± 10 | Fumed silica with surface | 18.8 ± 10 |
| area of 200 m²/gram | | area of 200 m²/gram | |
| Platinum catalyst complex | 0.06 | Trimethyl endblockedmethyl- | 3.8 ± 3 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.015 |
| | | 1,3,5,7-tetravinyl- | (between |
| | | cyclotetrasiloxane | 0.001-0.05) |
| | | Tetrapropoxysilane | 2.3 ± 2 |
| | | adhesion promoter | |

TABLE 8

| Part 1 | | Part 2 | |
|---|---|---|---|
| Component | Wt % | Component | Wt % |
| Vinyl endblockedpolydimethyl | 79.95 ± 20 | Vinyl endblocked polydimethyl | 75.29 ± 20 |
| siloxane polymer | | siloxane polymer | |
| Fumed silica with surface | 19.99 ± 10 | Fumed silica with surface | 18.8 ± 10 |
| area of 200 m²/gram | | area of 200 m²/gram | |
| Platinum catalyst complex | 0.06 | Trimethyl endblockedmethyl- | 3.8 ± 3 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.015 |
| | | 1,3,5,7-tetravinyl- | (between |
| | | cyclotetrasiloxane | 0.001-0.05) |
| | | N-(triethoxysilylpropyl)-O- | 0.37 |
| | | polyethylene oxide | (0.1-2) |
| | | urethane adhesion | |
| | | promoter | |

TABLE 9

| Part 1 | | Part 2 | |
|---|---|---|---|
| Component | Wt % | Component | Wt % |
| Vinyl endblockedpolydimethyl | 79.95 ± 20 | Vinyl endblockedpolydimethyl | 75.29 ± 20 |
| siloxane polymer | | siloxane polymer | |
| Fumed silica with surface | 19.99 ± 10 | Fumed silica with surface | 18.8 ± 10 |
| area of 200 m²/gram | | area of 200 m²/gram | |
| Platinum catalyst complex | 0.06 | Trimethyl endblockedmethyl- | 3.8 ± 3 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.015 |
| | | 1,3,5,7-tetravinyl- | (between |
| | | cyclotetrasiloxane | 0.001-0.05) |
| | | Tetrapropoxysilane | 2.57 ± 2.5 |
| | | adhesion promoter | |

TABLE 10

| Part 1 | | Part 2 | |
|---|---|---|---|
| Component | Wt % | Component | Wt % |
| Vinyl endblockedpolydimethyl | 79.95 ± 20 | Vinyl endblockedpolydimethyl | 75.29 ± 20 |
| siloxane polymer | | siloxane polymer | |
| Fumed silica with surface | 19.99 ± 10 | Fumed silica with surface | 18.8 ± 10 |
| area of 200 m²/gram | | area of 200 m²/gram | |
| Platinum catalyst complex | 0.06 | Trimethyl endblockedmethyl- | 3.8 ± 3 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.015 |
| | | 1,3,5,7-tetravinyl- | (between |
| | | cyclotetrasiloxane | 0.001-0.05) |
| | | N-(triethoxysilylpropyl)-O- | 2.57 ± 2.5 |
| | | polyethylene oxide | |
| | | urethane adhesion | |
| | | promoter | |

Table 11 shows an exemplary formulation including a thixotropic agent added to make the formulation non-slump at a concentration of between 0.25-3 wt %. The formulation of Table 10 is contemplated to have comparable work times (e.g., between 10-90 seconds), setting times (e.g., between 1-10 minutes), adhesion properties, hardness, tensile strength, and elasticities at break as the formulation of Table 1.

TABLE 11

| Part 1 | | Part 2 | |
|---|---|---|---|
| Component | Wt % | Component | Wt % |
| Vinyl endblockedpolydimethyl | 79.32 ± 20 | Vinyl endblockedpolydimethyl | 74.9 ± 20 |
| siloxane polymer | | siloxane polymer | |
| Fumed silica with surface | 19.83 ± 10 | Fumedsilica with surface | 18.7 ± 10 |
| area of 200 m²/gram | | area of 200 m²/gram | |
| Platinum catalyst complex | 0.06 | Trimethyl endblockedmethyl- | 3.7 ± 3 |
| | (0.001-0.2) | hydrogen siloxane polymer | |
| | | crosslinker (containing 50% | |
| | | methyl hydrogen and 50% dimethyl) | |
| Hydroxyl endblockedpolydimethyl | 0.79 | 1.3.5.7-tetramethyl- | 0.015 |
| siloxane (thixotrope) | (between | 1.3.5.7-tetravinyl- | (between |
| | 0.25-3) | cyclotetrasiloxane | 0.001-0.05) |
| | | Tetrapropoxysilane | 2.2 ± 2 |
| | | adhesion promoter | |
| | | N-(triethoxysilylpropyl)-O- | 0.37 |
| | | polyethylene oxide | (0.1-2) |
| | | urethane adhesion | |
| | | promoter | |

Table 12 shows another exemplary formulation including less platinum catalyst than the formulation of Table 1, which is contemplated to require a longer cure time.

TABLE 12

| Part 1 | | Part 2 | |
|---|---|---|---|
| Component | Wt % | Component | Wt % |
| Vinyl endblockedpolydimethyl | 79.97 ± 20 | Vinyl endblockedpolydimethyl | 74.9 ± 20 |
| siloxane polymer | | siloxane polymer | |
| Fumed silica with surface | 19.99 ± 10 | Fumed silica with surface | 18.7 ± 10 |
| area of 200 m²/gram | | area of 200 m²/gram | |
| Platinum catalyst complex | 0.04 | Trimethyl endblockedmethyl- | 3.7 ± 3 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.015 |
| | | 1,3,5,7-tetravinyl- | (between |
| | | cyclotetrasiloxane | 0.001-0.05) |
| | | Tetrapropoxysilane | 2.2 ± 2 |
| | | adhesion promoter | |
| | | N-(triethoxysilylpropyl)-O- | 0.37 |
| | | polyethylene oxide | (0.1-2) |
| | | urethane adhesion | |
| | | promoter | |

Table 13 shows another exemplary formulation including less crosslinker than the formulation of Table 1, which is contemplated to require a longer cure time.

TABLE 13

| Part 1 | | Part 2 | |
|---|---|---|---|
| Component | Wt % | Component | Wt % |
| Vinyl endblockedpolydimethyl | 79.95 ± 20 | Vinyl endblockedpolydimethyl | 76 ± 20 |
| siloxane polymer | | siloxane polymer | |
| Fumed silica with surface | 19.997 ± 10 | Fumed silica with surface | 79 ± 10 |
| area of 200 m²/gram | | area of 200 m²/gram | |
| Platinum catalyst complex | 0.06 | Trimethyl endblockedmethyl- | 2.3 ± 2 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.015 |
| | | 1,3,5,7-tetravinyl- | (between |
| | | cyclotetrasiloxane | 0.001-0.05) |
| | | Tetrapropoxysilane | 2.3 ± 2 |
| | | adhesion promoter | |
| | | N-(triethoxysilylpropyl)-O- | 0.38 |
| | | polyethylene oxide | (0.1-2) |
| | | urethane adhesion | |
| | | promoter | |

Table 14 shows another exemplary formulation including no fumed silica.

TABLE 14

| Component | Wt % | Component | Wt % |
|---|---|---|---|
| Vinyl endblockedpolydimethyl | 99.93 | Vinyl endblockedpolydimethyl | 92.1 ± 20 |
| siloxane polymer | | siloxane polymer | |
| Platinum catalyst complex | 0.07 | Trimethyl endblockedmethyl- | 4.6 ± 4 |
| | (0.001-0.2) | hydrogen siloxane polymer | |
| | | crosslinker (containing 50% | |
| | | methyl hydrogen and 50% dimethyl) | |
| | | 1,3,5,7-tetramethyl- | 0.018 |
| | | 1,3,5,7-tetravinyl- | (between |
| | | cyclotetrasiloxane | 0.001-0.05) |
| | | Tetrapropoxysilane | 2.8±2 |
| | | adhesion promoter | |
| | | N-(triethoxysilylpropyl)-O- | 0.46 |
| | | polyethylene oxide | (0.1-2) |
| | | urethane adhesion | |
| | | promoter | |

Exemplary formulations can also be viewed from a parts-per-hundred (pph) perspective. In Tables 15-17, all components are based on 100 parts of NuSil MED-4220 Part A or Part B (e.g., NuSil MED2-4220). Therefore, in order to add 3 pph of a component to 100 grams NuSil MED4220 Part A, 3 grams of the component would be added.

Table 15 shows an exemplary formulation having a working time of between 20-40 seconds (typically about 30 seconds), and a setting time of between 4-6 minutes (typically about 5 minutes) when Part 1 and Part 2 are mixed together and placed on skin.

TABLE 15

| Part | | Part | |
|---|---|---|---|
| Component | pph | Component | pph |
| NuSil MED4220 Part A | 100 | NuSil MED4220 Part B | 100 |
| NuSil CAT-50 | 0.01-0.30 | NuSil XL-100 | 0.5-5 |
| | (e.g., | | (e.g., |
| | 0.09) | | 1.5-2.5, |
| | | | or 2.0) |
| | | Gelest SIT 7777.0 | 0.5-6.0 |
| | | | (e.g., |
| | | | 2.0-4.0, |
| | | | or 3.0) |
| | | Gelest SIT 8192.0 | 0.01-1.0 |
| | | | (e.g., |
| | | | 0.5) |

Table 16 shows an exemplary formulation including radio-opaque particles for detection by X-ray.

TABLE 16

| Part 1 | | Part 2 | |
|---|---|---|---|
| Component | pph | Component | pph |
| NuSil MED4220 Part A | 100 | NuSil MED4220 Part B | 100 |
| NuSil MED2-4800 | 10.0-100.0 | NuSil XL-100 | 0.5-5 |
| | (e.g., | | (e.g., |
| | 20.0-60.0, | | 1.5-2.5, |
| | or 40.0) | | or 2.0) |
| NuSil CAT-50 | 0.01-0.30 | Gelest SIT 7777.0 | 0.5-6.0 |
| | (e.g., | | (e.g., |
| | 0.1) | | 2.0-4.0, |
| | | | or 3.0) |
| | | Gelest SIT 8192.0 | 0.01-1.0 |
| | | | (e.g., |
| | | | 0.5) |

Table 17 shows an exemplary formulation including pigments for, among other things, visibility, aesthetics (e.g., with designs), identification, or camouflaging (e.g., flesh tones, bright tones, or any other suitable tones).

TABLE 17

| Part 1 | | Part 2 | |
|---|---|---|---|
| Component | pph | Component | pph |
| NuSil MED4220 Part A | 100 | NuSil MED4220 Part B | 100 |
| NuSil MED2-4800 | 10.0-100.0 | NuSil XL-100 | 0.5-5 |
| | (e.g, | | (e.g, |
| | 20.0-60.0, | | 1.5-2.5, |
| | or 40.0) | | or 2.0) |
| NuSil CAT-50 | 0.01-0.30 | Gelest SIT 7777.0 | 0.5-6.0 |
| | (e.g., | | (e.g, |
| | 0.1) | | 2.0-4.0, |
| | | | or 3.0) |
| NuSil MED-4800-5 | 1.0-10.0 | Gelest SIT 8192.0 | 0.01-1.0 |
| | (e.g., | | (e.g, |
| | 3.0-70, | | 0.5) |
| | or 5.0) | | |

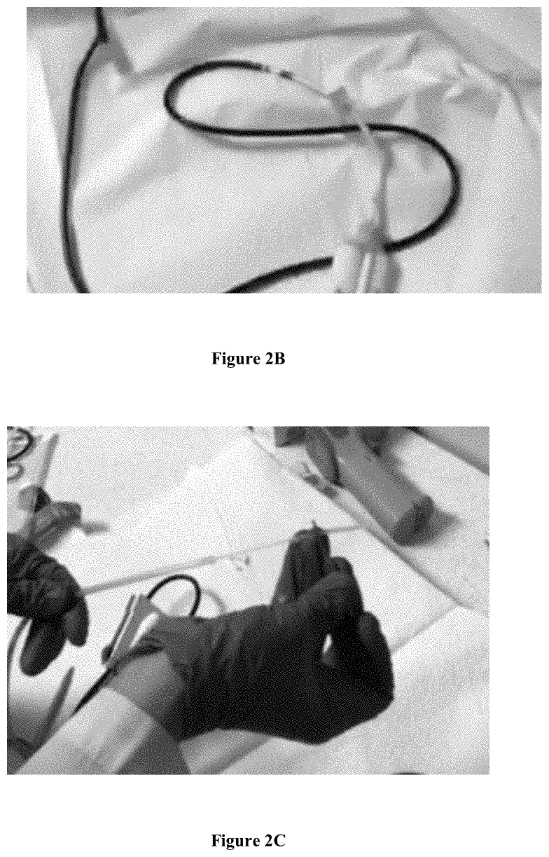

FIGS. 2A-2C exemplarily illustrate injection of contemplated compositions into a simulated blood vessel (silicon tubing as shown in FIG. 2A), polymerization to form a seal as shown in FIG. 2B, and removal in a single piece as shown in FIG. 2C.

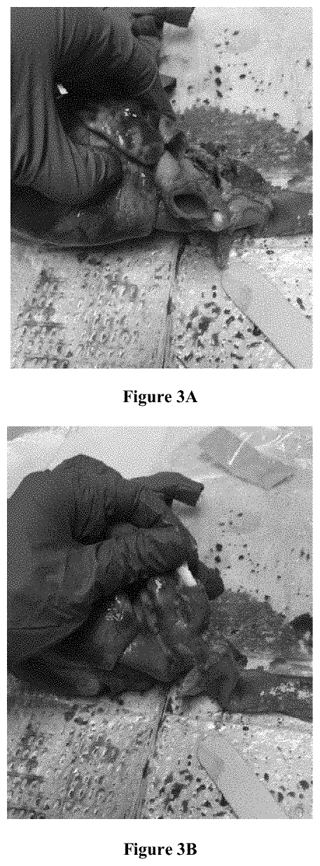

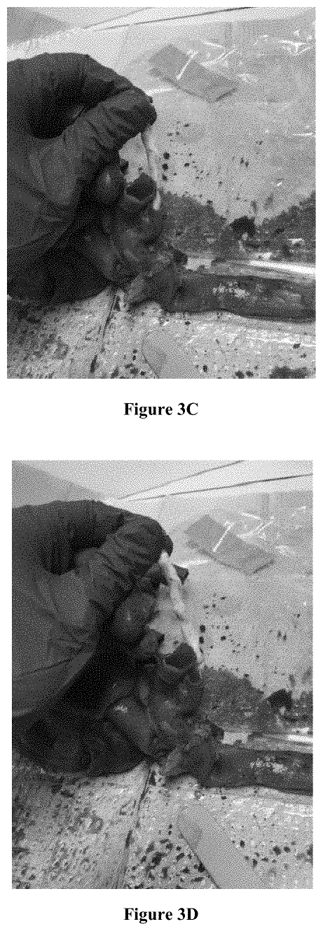

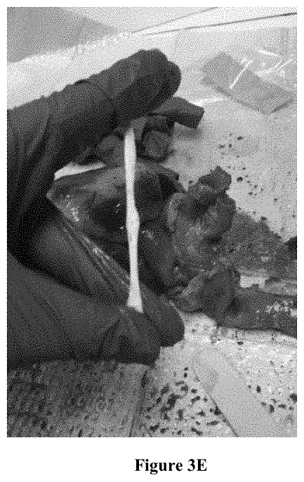

FIGS. 3A-3E illustrate the formulations used in FIGS. 2A-2C cured within and removed from a vessel of a pig's heart. The vessel is relatively large and has a smooth inner surface that largely lacks crevices. The formulation used did not substantially adhere to the inner surface, and was removed as a single piece. Where greater adhesion is desired, an alternative formulation having alternative or additional adhesion promoters could be used. Additionally or alternatively, a mesh stent could be placed within the target area of the vessel to provide a substrate the formulation could polymerize around and adhere to.

Therefore, it should be appreciated that the compositions as presented herein may advantageously be used for occlusion during embolism surgery, and particularly as replacement for ligatures or coils. Similarly, compounds according to the inventive subject matter may be used as replacement for n-butyl cyanoacrylate where blood vessels are to be sealed. Viewed from a different perspective, he compositions presented here are also suitable for transcatheter arterial chemoembolization. Most typically, and depending on the particular use, typical volumes deployed will be between 0.5 and 50 mL, and more typically between 1 mL and 20 mL. Consequently, seals formed in blood vessels may be relatively small (e.g., extending no more than 3 mm along the vessel), or longer (e.g., between 3 mm and 10 mm, and even longer). Furthermore, embolization may be temporary to assist in a surgical procedure and as such may be removed within a few hours, or longer lasting such as in tumor embolizations, ovarian arterial occlusion for animal sterilization, etc.

Where a base component is delivered to the target site, for example prior to applying the polymerizable composition that forms a seal, such base component could comprise at least one of an adhesion promoter, a catalyst, a volume expander, a temporary seal, a blood-based product, blood substitutes, a buffer solution, or a drug (e.g., a cancer drug). For example, the base component could act as or include a primer that promotes adhesion of the polymerizable composition to the vessel when applied, and include a catalyst to decrease the cure time of the polymerizable composition within the vessel. Additionally or alternatively, the base component can include a temporary seal component, such as LeGoo, which can temporarily block a movement of the polymerizable composition until completely cured. Once the polymerizable composition has cured to form a seal, the temporary block can be dissolved or otherwise removed. Preferably, the base component will not negatively impact the polymerizable composition's ability to adhere to inner wall of the vessel, or cure in situ in a short amount of time.

Contemplated adhesion promoters can include a silane coupling agent containing one or more functional groups that bond with the polymerizable composition or components thereof. Some contemplated adhesion promoters include a tetramethoxysilane, a tetraethoxysilane, a tetraisopropoxysilane, a tetrapropoxysilane, a tetrabutoxysilane, and a tetraacetoxysilane, a 3-aminopropropyltrimethoxysilane, tris(2-methoxyethoxy)(vinyl)silane, vinyltriethoxysilane, tetrakis(2-methoxyethyl)ester, and trimethoxy-7-octenylsilane.

When included, the adhesion promoter(s) can be present in the base component in any suitable amount. For example, it is contemplated that the adhesion promoter can be present in a liquid injectable base component in any suitable concentration, including for example, a concentration of between 0.1-100 mg/ml, between 0.1-75 mg/ml, between 0.1-50 mg/ml, between

0.1-10 mg/ml, or between 0.5-10 mg/ml.

Similarly to the polymerizable composition, the base component can also include a silicone or other catalyst that promotes curing of the polymerizable composition, which can be present in any suitable concentration (e.g., between 0.001-50 mg/ml).

Where the base component is a silicone primer, the primer will typically include one or more reactive silanes, a catalyst, and a solvent carrier (among other things). The reactive silanes can include a reactive group that is compatible with the polymerizable composition, and another reactive group that is compatible with the substrate (e.g., vessel) to thereby promote adhesion of the polymerizable composition to the substrate. One exemplary silicone primer comprises between 88-93 wt % isopropyl alcohol (e.g., 88 wt %), between 1-5 wt % tetrapropoxy silane (e.g., 3%), between 1-5 wt % titanium IV butoxide (e.g., 3%), and between 0.01-2 wt % platinum catalyst (e.g., 1 wt %). However, all suitable silicone primer compositions are contemplated.

Each of the base components and the polymerizable composition can be delivered to a target site within a vessel via a catheter. In some aspects of the inventive subject matter, the catheter can comprise or be coupled to a detachable tip. The detachable tip can comprise a pre formed implant, for example, an embolus that causes an embolism, or a stent-like tubular support that relieves an obstruction.

The pre-formed implant can be porous and include a plurality of holes through which the polymerizable composition can pass. Once the pre-formed implant is positioned at the target site, the components of the polymerizable composition can simultaneously or sequentially be delivered to the target site via the plurality of holes. Before, during or after the curing of the polymerizable composition at the target site and adherence of the polymerizable composition to the implant and the vessel through the plurality of holes, the implant can be detached from the catheter, and the catheter can be removed.

The pre-formed implant can be made from any suitable bio-compatible material, and can advantageously be made of a polymerizable composition of the inventive subject matter.

The PHOSITA should appreciate that different materials can be obtained from different commercial suppliers. For example, it is contemplated that components of some contemplated polymerizable compositions or base component can be obtained from commercial suppliers, for example, Silbond Corporation, Chemat, H.W. SandsCorp., Fluorochem USA, Gelest, Inc., Dupont Performance chemicals, Nusil Technology, Power Chemical Corporation, Rhodia Silicones, Reliance Silicones, or Zentek. The microcatheter can be obtained from, for example, Concentric Medical, Baylis Medical, Navilyst Medical, Asahi Intecc, or MicroVension.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Also, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

The discussion herein provides example embodiments of the inventive subject matter.

Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent, however, to those skilled in the art that many more modifications besides those already described are possible without departing from the inventive concepts herein. The inventive subject matter, therefore, is not to be restricted except in the spirit of the disclosure. One skilled in the art will recognize many methods and materials similar or equivalent to those described herein, which could be used in the practice of the present invention. Indeed, the present invention is in no way limited to the methods and materials described. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A method for delivering a polymerizable composition to a target site of a vessel, comprising:
delivering a first component of the polymerizable composition to the target site, the first component including a siloxane polymer and a catalyst;
delivering a second component of the polymerizable composition to the target site, the second component including a silicone polymer and a crosslinker;
wherein at least one of the first and second components include at least one of a pigment and radio-opaque particles to aid in visualizing the polymerizable composition within the vessel; and
wherein the first and second components, when combined, are formulated to polymerize in situ to form a seal within 5 minutes.

2. The method of claim 1, wherein at least one of the first and second components comprises a thixotropic agent.

3. The method of claim 1, wherein the polymerizable composition has a work time of less than 60 seconds.

4. The method of claim 1, wherein at least one of the first and second components comprises a first adhesion promoter to increase an adhesion of the polymerizable composition to an inner wall of the vessel.

5. The method of claim 4, wherein at least one of the first and second components comprises a second adhesion promoter, wherein the first and second adhesion promoters are present in a synergistic amount with respect to the adhesion of the polymerizable composition to the inner wall of the vessel.

6. The method of claim 1, wherein the seal has a hardness of between 0 on the Shore 00 durometer scale and 40 on the Shore A durometer scale.

7. The method of claim 1, wherein the seal has a hardness of between 10 on the Shore 00 durometer scale and 30 on the Shore A durometer scale.

8. The method of claim 1, wherein the seal has a hardness of between 15-25 on the Shore A durometer scale.

9. The method of claim 1, wherein the seal has an elongation at break of between 120-1000%.

10. The method of claim 1, wherein the seal has an elongation at break of between 120-200%.

11. The method of claim 1, wherein at least one of the first and second elastomer components comprises fumed amorphous silica having a surface area of between 150-250 m²/gram.

12. The method of claim 1, wherein the catalyst comprises a platinum catalyst, and wherein the crosslinker comprises a methyl hydrogen crosslinker.

13. The method of claim 1, wherein at least one of the first and second elastomer components comprises polydimethyl siloxane as a thixotropic agent.

14. The method of claim 1, wherein a combination of the first and second components comprises between 60-90 wt % of the siloxane polymer, between 10-30 wt % of a fumed silica, between 0.06-0.3 wt % of the catalyst, and between 2-10 wt % of the crosslinker.

15. The method of claim 1, wherein the siloxane polymer comprises a vinyl endblocked polydimethyl siloxane polymer.

16. The method of claim 15, wherein the vinyl endblocked polydimethyl siloxane polymer has a chain length of between 300-400 repeating units, and a molecular weight of between 20,000-32,000 Daltons.

17. The method of claim 1, wherein the at least one of the first and second components comprises radio-opaque particles, and wherein the radio-opaque particles comprises barium sulfate, or wherein the at least one of the first and second components comprises pigments, and wherein the pigments have a size of less than 20 microns

18. The method of claim 1, wherein the seal is formulated to adhere to an inner wall of the vessel for a period of at least 12 hours.

19. The method of claim 1, wherein the seal formed within 5 minutes of combining the first and second components, comprises a stent formed in situ at the target site.

20. The method of claim 1, wherein the first and second components are delivered to the target site via a catheter, and wherein the seal is formulated to not adhere to an outer surface of the catheter.
